(19) United States
(12) Patent Application Publication
(10) Pub. No.: US 2008/0181913 A1
(43) Pub. Date: Jul. 31, 2008

(54) Sugar immunogens

(75) Inventors: Raymond Allen Dwek, Oxford (GB); Christopher Scanlan, Oxford (GB); David Cameron Dunlop, Kearsley (AU); Fatma MH Mansab, London (GB); Sarah Erin Tully, San Diego, CA (US); Paul Wentworth, San Diego, CA (US); Nicole Zitzmann, Oxford (GB)

Correspondence Address: FOLEY AND LARDNER LLP;SUITE 500, 3000 K STREET NW, WASHINGTON, DC, 20007, US

(73) Assignee: United Therapeutics Corporation

(21) Appl. No.: 12/010,654

(22) Filed: Jan. 28, 2008

Related U.S. Application Data
(60) Provisional application No. 60/887,033, filed on Jan. 29, 2007.

(51) Int. Cl. A61K 39/21; A61P 31/12

(52) U.S. Cl. 424/208.1

(57) ABSTRACT

Disclosed are compositions and methods useful for inducing an immunogenic response in a subject or host. In particular, the compositions and methods may be directed to carbohydrate HIV vaccines and to methods of producing a carbohydrate HIV vaccine by introducing antigenic sugars into mimics of the glycans of the HIV envelope glycoproteins gp120 and gp41.

### RELATED APPLICATIONS

The present application claims priority to U.S. provisional application No. 60/887,033 filed on Jan. 29, 2007 to Raymond Dwek et al., which is incorporated herein by reference in its entirety.

### FIELD

The present inventions relate generally to the field of sugar immunogens.

### BACKGROUND

Anti-carbohydrate recognition represents a major component of both adaptive and innate immunity. However, only in a limited number of cases has the protective nature of antibodies to surface carbohydrates been exploited in a vaccine design.

The antigenic role of glycosylation is of particular significance in the case of human immunodeficiency virus type 1 (HIV-1). The surface of HIV-1 is covered by large, flexible and poorly immunogenic N-linked carbohydrates that form an ‘evolving glycan shield’ that promotes humoral immune evasion (see, e.g., X. Wei et. al. “Antibody neutralization and escape by HIV-1”, Nature, 422(6929), pp. 307-312, 2003, incorporated hereby by reference in its entirety). Three major explanations for the poor immunogenicity of HIV glycans have been proposed. Firstly, the glycans attached to HIV are synthesized by the host cell and are, therefore, immunologically ‘self’. Secondly, the binding of a protein to a carbohydrate is generally weak and, thus, limiting the potential for high affinity anti-carbohydrate antibodies. Finally, multiple different glycoforms can be attached to any given N-linked attachment site, thus, producing a highly heterogeneous mix of potential antigens. A wide range of complex, oligomannose and hybrid type glycans are all present on HIV, with the oligomannose glycans tightly clustered on the exposed outer domain of gp120. However, antibodies to HIV carbohydrates are not normally observed during infection.

The HIV-1 gp120 molecule is extensively N-glycosylated with approximately half the molecular weight of this glycoprotein contributed by covalently attached N-glycans. The crystal structure of the gp120 core with N-glycans modeled onto the glycoprotein surface identifies one face of the gp120 molecule that contains a cluster of N-glycans (see, e.g., P. D. Kwong et. al. “Structure of an HIV gp120 envelope glycoprotein in complex with the CD4 receptor and a neutralizing human antibody”, Nature, 393(6686) pp. 648-659, 1998, incorporated hereby by reference in its entirety). This face has been denoted the immunologically silent face because only one antibody (2G12) able to recognize this region of the glycoprotein molecule has been identified so far. The N-glycosylation of the HIV-1 gp120 molecule is thought to play a major role in immune evasion by preventing antibody accessibility to antigenic protein epitopes that lie underneath the N-glycosylation sites. In this instance, the exact structures of the N-glycans are of little importance provided they shield the underlying gp120 molecule from antibody recognition. Thus, the gp120 glycan shield can evolve by the introduction of new N-glycosylation sites following mutation of the viral genome. This promotes continued evasion of host immunity.

Although antibodies to carbohydrates of HIV are rare, there are many other pathogens, whose carbohydrate moieties elicit a strong antibody response. Indeed, a notable feature of the human humoral anti-carbohydrate reactivity is the widespread existence of anti-mannose antibodies, specific for α1→2 linked mannose oligosaccharides. Unlike 2G12, however, these antibodies do not bind to mannose that is presented within the context of ‘self’ oligomannose glycans. The probable targets of the natural anti-mannose antibodies are the cell wall mannans present on the lipids and proteins of many commonly occurring yeasts. Immunization with yeast mannans can provide some humoral cross-reactivity with gp120 carbohydrates (see, e.g., W. E. Muller et. al. “Polyclonal antibodies to mannan from yeast also recognize the carbohydrate structure of gp120 of the AIDS virus: an approach to raise neutralizing antibodies to HIV-1 infection in vitro”, AIDS. 1990 February;4(2), pp. 159-62., incorporated hereby by reference in its entirety; and W. E. Muller et. al. “Antibodies against defined carbohydrate structures of Candida albicans protect H9 cells against infection with human immunodeficiency virus-I in vitro”, J Acquir Immune Defic Syndr. 1991;4(7) pp. 694-703, incorporated hereby by reference in its entirety). However, the titers and affinities observed are not sufficient to warrant use as a prophylactic.

The above notwithstanding, one rare, neutralizing anti-gp120 antibody, 2G12, does bind to a specific carbohydrate epitope on the HIV envelope. The epitope recognized by 2G12 is a highly unusual cluster of mannose residues, present on the outer domain of gp120 (see, e.g., C. N. Scanlan et. al. “The Broadly Neutralizing Anti-Human Immunodeficiency Virus Type 1 Antibody 2G12 Recognizes a Cluster of α1->2 Mannose Residues on the Outer Face of gp120 J. Virol. 76 (2002) 7306-7321, incorporated hereby by reference in its entirety). The primary molecular determinant for 2G12 binding is the α1→2 linked mannose termini of the glycans attached to Asn332 and Asn392 of gp120. This cluster, although consisting of ‘self’ glycans is arranged in a dense array, highly atypical of mammalian glycosylation, thus, providing a structural basis for ‘non-self’ discrimination by 2G12. Structural studies of the 2G12 Fab reveal that the two heavy chains of the Fab are interlocked via a previously unobserved domain-exchanged configuration (see, e.g., D. Calarese et. al. “Antibody domain exchange is an immunological solution to carbohydrate cluster recognition”, Science, vol. 300, pp. 2065-2071, 2003, incorporated hereby by reference in its entirety). The extended paratope, formed by this domain exchanged Fab, provides a large surface for the high avidity binding of multivalent carbohydrates.

Passive transfer studies of 2G12 indicate that this antibody can protect against viral challenge in animal models of HIV-1. The molecular basis has been elucidated for the broad specificity of 2G12 against a range of HIV-1 primary isolates. Therefore, based on the known structure of the 2G12 epitope, it is highly desirable to develop an immunogen that can be capable of eliciting 2G12-like antibodies and can contribute to sterilizing immunity against HIV-1. However, the design of such an immunogen has to overcome both the structural constraints required for antigenic mimicry of the glycan epitope on gp120 and the immunological constraints inherent to the poorly immunogenic N-linked glycans of HIV.

One approach to gp120 immunogen design is to synthetically recreate the antigenic portion of gp120 to which 2G12 binds (see, e.g., H. K Lee et. al. “Reactivity-Based One-Pot Synthesis of Oligomannoses: Defining Antigens Recognized by 2G12, a Broadly Neutralizing Anti-HIV-1 Antibody”, Angew. Chem. Int. Ed. Engl, 43(8), pp. 1000-1003, 2004, incorporated hereby by reference in its entirety; H. Li et. al. “Design and synthesis of a template-assembled oligomannose cluster as an epitope mimic for human HIV-neutralizing antibody 2G12”, Org. Biomol. Chem., 2 (4), pp. 483-488, 2004 incorporated hereby by reference in its entirety; L.-X. Wang, “Binding of High-Mannose-Type Oligosaccharides and Synthetic Oligomannose Clusters to Human Antibody 2G12: Implications for HIV-1 Vaccine Design”, Chem. Biol. 11(1), pp. 127-34, 2004, incorporated hereby by reference in its entirety). Other approaches for preparing carbohydrate immunogens are described in U.S. 2006-0251680, which is incorporated herein by reference. Presentation of synthetic mannosides in a multivalent format can increase their affinity to 2G12 by almost 100-fold (see, e.g., L.-X. Wang, “Binding of High-Mannose-Type Oligosaccharides and Synthetic Oligomannose Clusters to Human Antibody 2G12: Implications for HIV-1 Vaccine Design”, Chem. Biol. 11(1), pp. 127-34, 2004).

Although the synthetic approach to immunogen design is a potentially powerful one, there are significant challenges to the ‘rational’ design of immunogens. It is highly desirable to develop alternative methods of designing carbohydrate immunogens.

### SUMMARY

Disclosed are pharmaceutical compositions and kits for inducing an immunogenic response against an antigen that comprises an oligo-D-mannose moiety. Also disclosed are methods of using the compositions for inducing an immunogenic response and methods for making the pharmaceutical compositions. Typically, the pharmaceutical compositions include: (a) an effective concentration of an antigen comprising a substituted oligo-D-mannose moiety in which at least one D-mannose residue of the oligo-D-mannose moiety of the antigen is substituted by at least one non-D-mannose monosaccharide residue; and (b) a carrier (e.g., an excipient, diluent, and/or an adjuvant).

In some embodiments, the non-D-mannose monosaccharide residue comprises a structural mimic or analogue of D-mannose. The non-D-mannose monosaccharide residue may comprise a monosaccharide residue that is antigenic in a subject to which the pharmaceutical composition is administered (e.g., a human). The non-D-mannose monosaccharide residue may comprise a monosaccharide residue that is non-naturally produced or observed in the subject (e.g., a human). The non-D-mannose monosaccharide residue may comprise a D- or L-type monosaccharide. Typically, the non-D-mannose monosaccharide residue has five- or six-carbons and is optionally substituted at a carbon or hydroxyl position. Examples of non-D-mannose monosaccharide residues may include monosaccharide residues selected from the group consisting of deoxy-monosaccharides (e.g., rhamnose), halo-substituted monosaccharides or halo-substituted deoxy-monosaccharides (e.g. 6-deoxy-6-fluoro-D-glucose), nitro-substituted monosaccharides, amino-substituted monosaccharides (e.g., nojirimycin and deoxynojirimycin), sulfo-substituted monosaccharides, phosphor-substituted monosaccharides, and aryl-substituted monosaccharides (e.g, 1-paranitrophenyl-D-rhamnose).

In the pharmaceutical composition, the substituted oligo-D-mannose moiety may be present as part of a larger molecule such as a glycoprotein, a glycoconjugate scaffold, or a dendrimer. In some embodiments, the substituted oligo-D-mannose moiety is present in the pharmaceutical composition as part of a glycoprotein where the substituted oligo-D-mannose moiety is linked as an N-glycan.

The oligo-D-mannose moiety may include a straight chain or branched oligo-D-mannose oligosaccharide. In some embodiments, the oligo-D-mannose moiety includes about 5-12 mannose residues (e.g., Man9GlcNAc2, Man8GlcNAc2, Man7GlcNAc2, or Man6GlcNAc2).

The mannose residues of the oligo-D-mannose moiety may be linked via a reducing hydroxyl and any other suitable hydroxyl group. In some embodiments, the mannose residues of the oligo-D-mannose moiety may be linked via 1→2 linkages (e.g., α1→2 linkages), via 1→3 linkages (e.g., α1→3 linkages), via 1→6 linkages (e.g., α1→6 linkages), or a combination thereof. The non-D-mannose monosaccharide residue of the substituted oligo-D-mannose moiety may be linked via any suitable linkage (e.g., via an α1→2 linkage, via an α1→3 linkage, or via an α1→6 linkage, preferably an α1→2 linkage).

The oligo-D-mannose moiety may be linked to a polypeptide. For example, the oligo-D-mannose moiety may be linked as a N-glycan where the oligo-D-mannose moiety may include one or more N-acetylgalactosamine residues (GlcNAc) which are linked to a polypeptide (e.g., at an asparagine residue (Asn) via an amide linkage). Exemplary N-glycans may include Man9GlcNAc2, Man8GlcNAc2, or Man7GlcNAc2.

The oligo-D-mannose moiety of the antigen may be substituted with any suitable non-D-mannose monosaccharide residue. In some embodiments, the oligo-D-mannose moiety is Man9GlcNAc2 and the substituted oligo-D-mannose moiety is Rham1Man8GlcNAc2.

The oligo-D-mannose moiety may have a structure represented according to the formula:

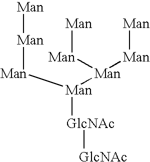

where “Man” is mannose, “GlcNAc” is N-acetylgalactosamine. Optionally, the oligo-D-mannose moiety is conjugated to a peptide (e.g., at an asparagine residue) by the terminal GlcNAc residue.

The substituted oligo-D-mannose moiety may have a structure represented according to one of the formulas:

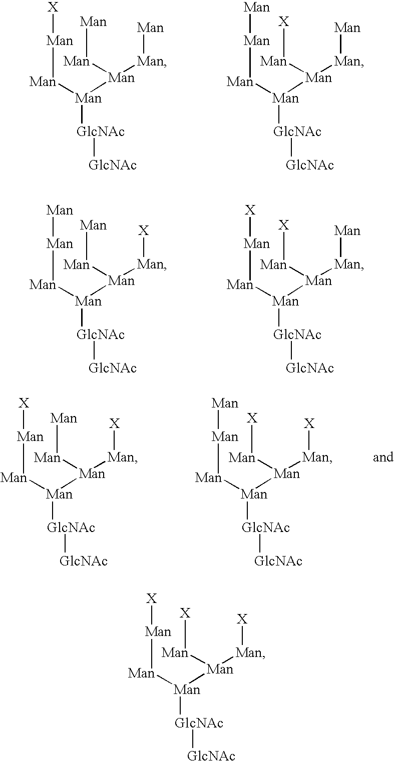

where “Man” is mannose, “GlcNAc” is N-acetylgalactosamine, and “X” is a non-D-mannose monosaccharide residue as described herein (e.g., rhamnose).

The antigen may be an HIV glycoprotein or fragment thereof having ten (10) or more contiguous amino acids linked to an oligo-D-mannose moiety (e.g., Man9GlcNAc2). The antigen may include HIV glycoprotein 120 (gp120) or HIV glycoprotein 41 (gp41). In some embodiments, the HIV glycoprotein is gp120 and the oligo-D-mannose moiety is the oligo-D-mannose moiety attached as an N-glycan at Asn332 or Asn392.

The composition typically comprises an effective amount of the antigen for inducing an immunogenic response in a subject (e.g., a human). In some embodiments, the composition is used to induce a humoral response in a subject, where the humoral response includes production of antibodies that specifically bind the oligo-D-mannose moiety.

Also disclosed are methods and kits for inducing an immunogenic response against an antigen that comprises an oligo-D-mannose moiety. Typically, the methods include administering any of the pharmaceutical compositions disclosed herein to a subject in need thereof (e.g., a human having or at risk for acquiring an HIV infection).

Also disclosed are methods for preparing an antigen that comprises a substituted oligo-D-mannose moiety, which optionally may be used for inducing an immunogenic response against an antigen comprising the non-substituted oligo-D-mannose moiety. Typically, the methods include: (a) treating an antigen that comprises an oligo-D-mannose moiety with a first glycosidase to remove at least one D-mannose residue; and (b) reacting the treated antigen with at least one non-D-mannose monosaccharide residue in the presence of a second glycosidase to provide the antigen that comprises the substituted oligo-D-mannose moiety.

The oligo-D-mannose moiety may include the oligo-D-mannose moiety present in HIV gp120 or HIV gp41. For example, the oligo-D-mannose moiety may include the N-glycan attached to Asn332 or Asn392 of HIV gp120. The oligo-D-mannose moiety of the method of preparation may include Man9GlcNAc2, Man8GlcNAc2, or Man7GlcNAc2.

In the method of preparation, the first glycosidase or the second glycosidase may be a mannosidase. Glycosidases may include exomannosidases and endomannosidases. Exemplary mannosidases include Class I endoplasmic reticulum (ER) mannosidase and Jack Bean mannosidase. In some embodiments, the first glycosidase is an exomannosidase and the second glycosidase is Jack Bean mannosidase. Suitable mannosidases may include retaining enzymes where the alpha- or beta-anomeric configuration of a saccharide is retained by the enzyme after the enzyme hydrolyzes a glycosidic bond. Suitable mannosidase may include inverting enzymes where the alpha- or beta-anomeric configuration of a saccharide is inverted to a beta- or alpha-anomeric configuration by the enzyme after the enzyme hydrolyzes a glycosidic bond.

For example, the methods of preparation may include use of mannosidase that is a retaining enzyme (e.g., Jack Bean mannosidase) where the non-D-mannose residue has an alpha-anomeric configuration. In other embodiments, the methods of preparation may include use of a mannosidase that is an inverting enzyme (e.g., Class I ER exomannosidase) where the non-D-mannose residue has a beta-anomeric configuration.

The methods of preparation may utilize any suitable non-D-mannose monosaccharide residue. In some embodiments, the non-D-mannose monosaccharide residue comprises a structural mimic or analogue of D-mannose. The non-D-mannose monosaccharide residue may include a monosaccharide residue that is antigenic in a subject (e.g., a human). In some embodiments, the non-D-mannose monosaccharide residue is a monosaccharide residue that is non-naturally produced or observed in the subject (e.g., a human). For example, the non-D-mannose monosaccharide residue may include a monosaccharide residue selected from the group consisting of deoxy-monosaccharides (e.g., rhamnose), halo-substituted monosaccharides, nitro-substituted monosaccharides, amino-substituted monosaccharides (e.g., nojirimycin and deoxynojirimycin), sulfo-substituted monosaccharides, phosphor-substituted monosaccharides, and paranitrophenyl-substituted monosaccharides.

In the methods of preparation, the antigen that comprises the oligo-D-mannose moiety may include a glycoprotein, a glycoconjugate scaffold, or a dendrimer. In some embodiments of the methods of preparation, the antigen that comprises the oligo-D-mannose moiety is a glycoprotein wherein the oligo-D-mannose moiety is linked as an N-glycan. The antigen may be an HIV glycoprotein (e.g., gp120 or gp41) or a fragment thereof having ten (10) or more contiguous amino acids linked to an oligo-D-mannose moiety. In some embodiments of the methods of preparation, the antigen is HIV gp120 and the oligo-D-mannose moiety is attached as an N-glycan at Asn332 or Asn392.

In some embodiments of the methods of preparation, the oligo-D-mannose moiety may include Man9GlcNAc2, Man8GlcNAc2, or Man7GlcNAc2. The non-D-mannose monosaccharide moiety may have a substitution at a hydroxyl position. For example, the non-D-mannose monosaccharide moiety may include an oxygen→hydrogen substitution at the C6 position (e.g., 6-deoxy-alpha-D-mannose or “rhamnose”). The substitution at a hydroxyl position may include a leaving group substitution (e.g., a nitrophenyl group at the C6 hydroxyl. The non-D-mannose monosaccharide residue may include paranitrophenyl-alpha-D-rhamnose. Exemplary prepared antigens may comprise substituted oligo-D-mannose moieties such as Rham1Man8GlcNAc2, Rham1Man7GlcNAc2, Rham1Man6GlcNAc2, and Rham1Man5GlcNAc2.

### BRIEF DESCRIPTION OF THE DRAWINGS

FIG. 1 provides an analysis of binding of human sera (n=10) to a glycan microarray measured by fluorescent anti-human antibodies.

FIG. 2 provides an example scheme for the synthesis of an antigenic derivate of oligomannose glycan.

FIG. 3 illustrates MALDI-TOFF mass spectrometric analysis of reaction products of the reaction of FIG. 2.

### DETAILED DESCRIPTION

The present disclosure is directed to pharmaceutical compositions, methods, and kits. In particular, the present disclosure relates to carbohydrate vaccines or immunogenic composition, methods for inducing antibodies against carbohydrate moieties, and immunogenic compositions and methods of producing them. In some embodiments, the present disclosure relates to carbohydrate HIV vaccines and immunogenic compositions and methods of producing them.

### RELATED APPLICATIONS

The present disclosure incorporates by reference in its entirety U.S. patent application Ser. No. 11/376,549, which was published as publication US 2006-0251680.

### DEFINITIONS

Unless otherwise specified, the terms “a” or “an” mean “one or more.”

Unless otherwise specified, the term “alkyl” as used herein refers to straight- and branched-chain alkyl radicals containing one or more carbon atoms and includes, for example, methyl, ethyl, butyl, and nonyl.

The term “aryl” as used herein refers to a monocyclic aromatic group such as phenyl or a benzo-fused aromatic group such as indanyl, naphthyl, or fluorenyl and the like.

The term “heteroaryl” refers to aromatic compounds containing one or more hetero atoms. Examples include pyridyl, furyl, and thienyl or a benzofused aromatic containing one or more heteroatoms such as indolyl or quinolinyl.

The term “heteroatom” as used herein refers to non-carbon atoms such as N, O, and S.

The term “cycloalkyl” as used herein refers to a carbocyclic ring containing 3, 4, 5, 6, 7, or 8 carbons and includes, for example, cyclopropyl and cyclohexyl.

Unless otherwise specified, the term “alkoxy” as used herein refers to a straight- or branched-chain alkoxy containing one or more carbon atoms and includes, for example, methoxy and ethoxy.

The term “alkenyl” as used herein refers to a straight or branched-chain alkyl containing one or more double bonds such as ethenyl and propenyl.

The term “aralkyl” as used herein refers to an alkyl substituted with an aryl such as benzyl and phenethyl.

The term “alkynyl” as used herein refers to a straight or branched-chain alkyl containing one or more triple bonds such as ethynyl and propynyl.

The term “aryloxy” as used herein refers to a substituent created by replacing the hydrogen atom in an —OH group with an aryl group, and includes, for example, phenoxy.

The term “aralkoxy” as used herein refers to an alkoxy group substituted with an aryl group, such as 2-phenylethoxy.

The term “alkylamino” as used herein refers to an amino group substituted with one alkyl group such as methylamino (—NHCH₃) and ethylamino (—NHCH₂CH₃). The term “dialkylamino” as used herein refers to an amino group substituted with two alkyl groups such as dimethylamino (—N(CH₃)₂) and diethylamino (—N(CH₂CH₃)₂).

The term “halogen” or “halo-substitution” refers to fluorine, chlorine, bromine or iodine.

A monosaccharide is any carbohydrate, such as tetroses, pentoses, and hexoses, that cannot be broken down to simpler sugars by hydrolysis. Non-D-mannose monosaccharides which may used in this invention include, but not limited to, residues derived from D- and L-type natural monosaccharides including 6-deoxysaccharides such as rhamnose, fucose, digitoxose, oleandrose and quinovose, hexoses such as allose, altrose, glucose, gulose, idose, galactose and talose, pentoses such as ribose, arabinose, xylose and lyxose, tetroses such as erythrose and threose, aminosaccharides such as glucosamine and daunosamine, uronic acids such as glucuronic acid and galacturonic acid, ketoses such as psicose, fructose, sorbose, tagatose and pentulose, and deoxysaccharides such as 2-deoxyribose; residues derived from natural or synthetic pyranose and furanose saccharides; and saccharide residue derivatives in which hydroxy and/or amino groups in any of the above residues are protected or acylated or include a leaving group (i.e., —O-leaving group) or saccharides having a halogenated saccharide residue in which hydroxy is replaced with halogen such as fluorine. A leaving group in terms of “a leaving group of hydroxyl” means that which may be removed by an appropriate biochemical process such as hydrolysis. By the term “subject in need thereof” is in the present context meant a subject, which can be any animal, including a human being, in which an immunogenic response to the substituted oligo-D-mannose moiety brings about a therapeutic or preventive effect. A “subject in need thereof” may include a human who is infected with, or who is at risk for being infected with a pathogen such as human immunodeficiency virus type 1 or HIV-1. The term “subject” and “patient” and “host” are used herein interchangeably.

By the term “an effective amount” is meant an amount of the substance in question (e.g., an antigen comprising a substituted oligo-D-mannose moiety) which will in a majority of patients induce an immunogenic response (e.g., the production of antibodies against an antigen comprising the oligo-D-mannose moiety). The term “an effective amount” also implies that the substance is given in an amount which only causes mild or no adverse effects in the subject to whom it has been administered, or that the adverse effects may be tolerated from a medical and pharmaceutical point of view in the light of the severity of the disease for which the substance has been given for treatment or prevention.

As used herein, “treatment” includes both prophylaxis and therapy. Thus, in treating a subject, the compounds of the invention may be administered to a subject already harboring an infection or in order to prevent such infection from occurring.

In the present context the terms “a mannose analogue” or “a mannose mimic” should be understood, in a broad sense to mean any substance which mimics (with respect to binding characteristics) the mannose sugar which binds to an effective part of the 2G12 monoclonal antibody (available from the U.S. National Institute of Health (NIH) AIDS Research & Reference Reagent Program, catalog no. 1476). Thus, the analogue or mimic may simply be any other compound regarded as capable of mimicking the binding of a mannose sugar of a mannose-oligosaccharide to 2G12 antibody in vivo or in vitro. In the present context, the mannose analogue or mannose mimic exhibits at least one binding characteristic relevant for the binding of 2G12 antibody to HIV gp120. For example, in an analogue or mimic, each side chain could be replaced by another group having a similar stereochemistry or arrangement of polar and non-polar atoms, as long as any particular features which are essential for association with 2G12 antibody are preserved.

In some embodiments, the non-D-mannose monosaccharide has a formula selected from:

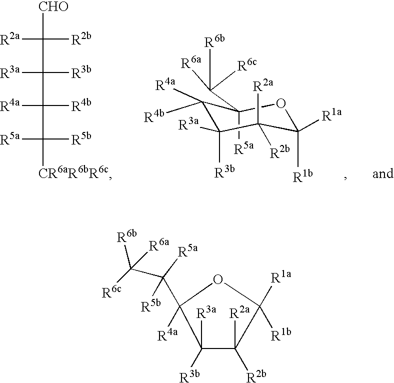

wherein each of R^{1a}, R^{1b}, R^{2a}, R^{2b}, R^{3a}, R^{3b}, R^{4a}, R^{4b}, R^{5a}, R^{5b}, R^{6a}, R^{6b}, and R^{6c}, is selected, independently from the other, from the group consisting of —H; —OH; —F; —Cl; —Br; —I; —NH₂; alkyl- and dialkylamino; linear or branched C₁₋₆ alkyl, C₂₋₆ alkenyl and alkynyl; aralkyl; linear or branched C₁₋₆ alkoxy; aryloxy; aralkoxy; -(alkylene)oxy(alkyl); —CN; —NO₂; —COOH; —COO(alkyl); —COO(aryl); —C(O)NH(C₁₋₆ alkyl); —C(O)NH(aryl); sulfonyl; (C₁₋₆ alkyl)sulfonyl; arylsulfonyl; sulfamoyl, (C₁₋₆ alkyl)sulfamoyl; (C₁₋₆ alkyl)thio; (C₁₋₆ alkyl)sulfonamide; arylsulfonamide; —NHNH₂; —NHOH; aryl; and heteroaryl. Each substiuent may be the same or different. In further embodiments, a carbon atom may be substituted with a heteroatom.

### Modification of Carbohydrates to Provide Immunogens

Although the synthetic approach to immunogen design is a potentially powerful one, the immunologically “self” oligomannose glycans are inherently poor immunogens. For example, the discrimination between “self” and “non self” mannosides is closely regulated, presenting a challenge to vaccine design. The carbohydrates that bind 2G12 (Man(α1-2)Man(α1-2)Man(α1-3)Man, D1 and [Man(α1-2)Man(α1-6)][Man(α1-2)Man(α1-3)]Man, D2D3) are naturally antigenic. However, these antigenic structures are nonetheless immunosilent within the context of a self glycan. At an atomic level, a single hydroxyl determines the antigenicity of monosaccharide α-D-mannose, compared to α-D-rhamnose. One approach is therefore to overcome immunological tolerance by the introduction of non-self, antigenic carbohydrates that maintain close structural homology to oligomannose glycans. For example, chemical and/or enzymatic modification of oligomannose glycans may produce antigenic mimics of the 2G12 epitope.

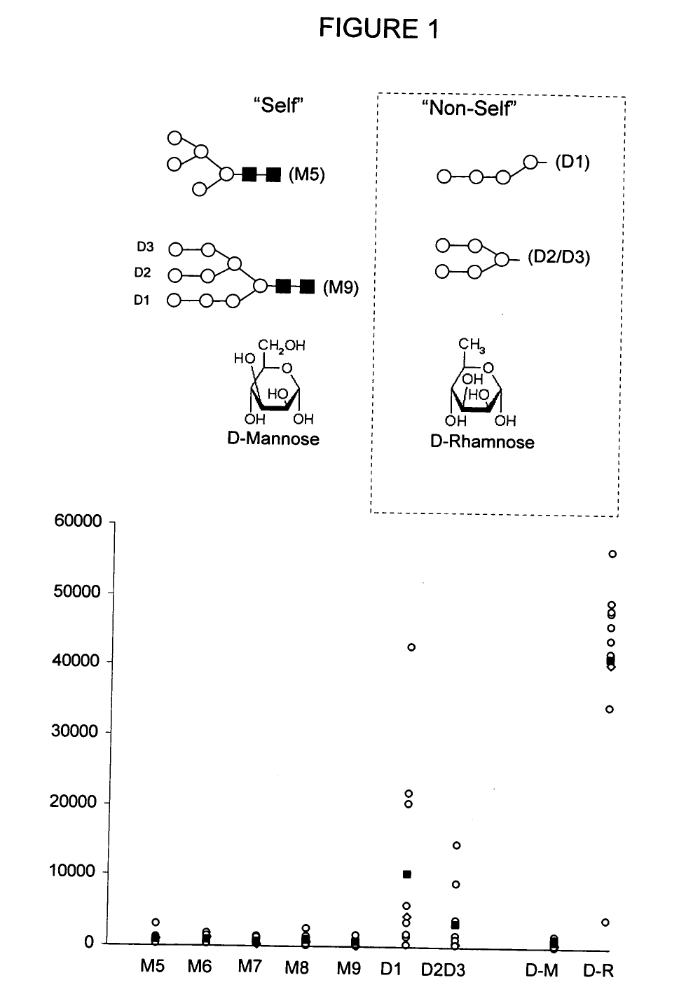

To identify carbohydrates that are naturally or inherently antigenic to the human immune system, one can screen human serum antibodies against immobilized glycans (see FIG. 1). This may reveal carbohydrates, and/or carbohydrate arrangements which are recognised as “non-self”. Analyses of human serum reactivity has revealed that alpha-D-rhamnose is such an antigenic sugar, whereas alpha-D-Mannose (the major constituent of the 2G12 epitope) is not (see FIG. 1). Significantly, rhamnose is a close structural mimic of mannose, differing only in lack of a single oxygen atom at the C6 position, and alpha-D-rhamnose is thus 6-deoxy-alpha-D-mannose. By incorporating rhamnose, or a similar antigenic mimics into the natural oligomannose structure of gp120, the inherent tolerance to the (mannose) sugars of HIV may be overcome.

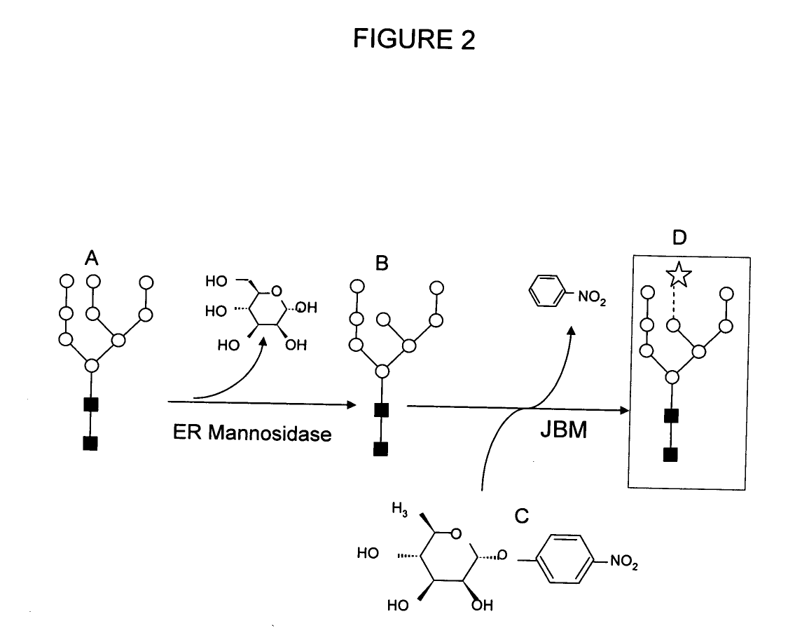

Thus, alpha-D-rhamnose (or other “non-self” monosaccharides) may be incorporated into the oligomannose glycans found on gp120. One method for synthesizing this antigenic glycan is to reverse the hydrolytic activity of mannosidases to yield mannose glycoconjugates. Thus an excess of a mannose analogue may be enzymatically transferred to a glycan by the action of alpha-mannosidases. A viable synthesis scheme is outlined in FIG. 2. The rhamnose-substituted glycan(s) retain binding sites for 2G12, which include the D1 and D3 arms of Man9GlcNAc2, but also contain a highly antigenic sugar (i.e., rhamnose). Such carbohydrate modifications are useful in the design of a carbohydrate vaccine for HIV-1.

### HIV Vaccines and Immunogenic Compositions

An HIV vaccine or immunogenic composition can be made by modifying an HIV component that comprises a carbohydrate moiety in such a way (e.g., modifying glycosylation) such that the modified HIV component becomes antigenic in a subject. The modified HIV component then can be administered to a subject to induce an immunogenic response such as production of antibodies that bind to the non-modified HIV component.

In the present context, the term “modifying glycosylation” or “modified glycosylation” means that glycans (oligosaccharides) of the component (e.g., a glycoprotein) differ by at least one and preferably by more than one from glycan from the glycans that are naturally found on the component.

The oligo-D-mannose moieties of the antigens disclosed herein may include high mannose glycans. High mannose glycans include glycans having at least one terminal Manα1,2Man linkage. Examples of such oligosaccharides are Man9GlcNAc2, Man8GlcNAc2, Man7GlcNAc2, Man6GlcNAc2 or their isomers. Preferably, the antigen is a glycoprotein having N-glycans and the N-glycans of the glycoprotein are predominantly Man9GlcNAc2 or its isomers.

### Self-Proteins for Presentation of Sugar Immunogens

The immune response to gp120 is normally dominated by antibodies specific to the protein core. The N-linked glycans do not usually play a direct role in antibody recognition. To eliminate both the immune response to, and the immune modulation by, the protein moiety, ‘self’ proteins can be employed as scaffolds for ‘non-self’ oligomannose clusters.

The expression of recombinant ‘self’ glycoproteins can provide a scaffold with oligomannose-type glycans, which mimic the 2G12 epitope. For example, recombinant ‘self’ glycoproteins may be modified to include modified glycosylation (e.g., a substituted oligo-D-mannose moiety). The advantage of this approach can be that the 2G12 epitope can be presented in an immunosilent, protein scaffold, with any antibody response directed only towards the substituted oligo-D-mannose moiety.

### Use of Modified Glycoproteins and Mannans as Immunogens

The present disclosure also provides an HIV vaccine or immunogenic composition comprising substituted oligo-D-mannose moieties having specific complementarity to the 2G12 antibody. Such substituted oligo-D-mannose moieties may be prepared from Mannans, which are polysaccharides containing mannose, preferably from yeast or bacterial cells. The mannans can be in the form of isolated mannans; whole yeast or bacterial cells, which may be killed cells or attenuated cells; or as mannans coupled to carrier molecule or protein. The mannans can be mannans for yeast or bacterial cells that a natural affinity to the 2G12 antibody. One example of such mannans can be mannan structures of Candida albicans that mimic the 2G12 epitope, i.e., have a natural specific complementarity to the 2G12 antibody. The mannan may be modified as describe herein to include one or more non-D-mannose monosaccharide residues.

The mannans can be also artificially or genetically selected mannans. Such mannans can be produced by iteratively selecting yeast or bacterial cells having a higher affinity to the 2G12 antibody. The starting pool of cells for this iterative process can comprise cells that exhibit some non-zero affinity or specificity. From the starting pool, a subset of cells can be selected that has a higher affinity to the 2G12 antibody than the rest of the cells. The cells of the subset can be then replicated and used as a starting pool for a subsequent iteration. Various criteria can be used for identifying a subset of cells having a higher affinity to the 2G12 antibody. For example, in a first iteration the cells that have a detectable affinity for the 2G12 antibody. In subsequent iterations, the selected cells can be cells representing The cells displaying a high affinity to the 2G12 antibody can selected out, using a fluorescence activated cell sorter (FACS), or by a direct enrichment using immobilized 2G12 for affinity separation. The selected mannan may be modified as described herein to include one or more non-D-mannose monosaccharide residues.

One non-limiting example that can be used for a starting pool of cells are S. cervisiae cells. The 2G12 antibody can bind S. cervisiae mannans, thus, indicating a certain non-zero degree of antigenic mimicry between mannans and gp120 glycoprotein. The carbohydrate structure of S. cerivisiae cell wall shares common antigenic structures with the oligomannose glycans of gp120. However, naturally occurring S. cervisiae mannans do not induce sufficient humoral cross reactivity to gp120 when used as a immunogen. The S. cervisiae mannans may be modified as described herein to include one or more non-D-mannose monosaccharide residues.

### Vaccines and Immunogenic Compositions

The pharmaceutical compositions disclosed herein may be used as a vaccine or immunogenic composition. The vaccine or immunogenic composition can be administered for vaccinating and/or immunogenizing against HIV of mammals including humans against HIV. The vaccine or immunogenic composition can include mannans (or modified mannans as described herein) having a specific complementarity to the 2G12 antibody and/or a glycoprotein prepared according to described methods above. The glycoprotein can be included in the vaccine as isolated or purified glycoprotein without further modification of its glycosylation.

The vaccine or immunogenic composition can be administered by any convenient means. For example, a glycoprotein and/or mannans (or modified glycoproteins or mannans) can administered as a part of pharmaceutically acceptable composition further contains any pharmaceutically acceptable carriers or by means of a delivery system such as a liposome or a controlled release pharmaceutical composition. The term “pharmaceutically acceptable” refers to molecules and compositions that are physiologically tolerable and do not typically produce an allergic or similar unwanted reaction such as gastric upset or dizziness when administered. Preferably, “pharmaceutically acceptable” means approved by a regulatory agency of the Federal or a state government or listed in the U.S. Pharmacopoeia or other generally recognized pharmacopoeia for use in animals, preferably humans. The term “carrier” refers to a diluent, adjuvant, excipient, or vehicle with which the compound is administered. Such pharmaceutical carriers can be sterile liquids, such as saline solutions, dextrose solutions, glycerol solutions, water and oils emulsions such as those made with oils of petroleum, animal, vegetable, or synthetic origin (peanut oil, soybean oil, mineral oil, or sesame oil). Water, saline solutions, dextrose solutions, and glycerol solutions are preferably employed as carriers, particularly for injectable solutions.

The vaccine or immunogenic composition can be administered by any standard technique compatible with glycproteins and/or mannans. Such techniques include parenteral, transdermal, and transmucosal, e.g., oral or nasal, administration.

### ILLUSTRATIVE EMBODIMENTS

The following not-limiting embodiments further illustrate the present invention.

Embodiment 1. A method of introducing antigenic sugars into oligomannose glycans to produce non-natural oligomannosides to improve the immunogenicity of the said oligomannose glycan.

Embodiment 2. The method of embodiment 1, wherein the immunogenicity of the non-natural oligomannose glycan relates to its ability to elicit anti-HIV antibodies.

Embodiment 3. The method of embodiment 1 or 2, wherein the sugar is identified as immunogenic through affinity binding studies of human sera to carbohydrates and carbohydrate arrays.

Embodiment 4. The method of any of embodiments 1-3, wherein the antigenic sugar is a structural mimic of D-mannose.

Embodiment 5. The method of any of embodiments 1-4, wherein the antigenic sugar is D-Rhamnose.

Embodiment 6. The method of any of embodiments 1-5, wherein the oligomannose glycans are Man9GlcNAc2, Man8GlcNAc2, Man7GlcNAc2, or structural analogues, mimics, or derivatives thereof.

Embodiment 7. The method of any of embodiments 1-6, wherein the oligomannose glycans, substituted according to embodiment 1 are arranged on the surface of a glycoprotein, glycoconjugate scaffold, or dendrimer.

Embodiment 8. The method of any of embodiments 1-7, wherein the introduction of antigenic sugars to oligomannose scaffold is achieved by condensation (reverse hydrolysis) using the catalytic activity of glycosidases.

Embodiment 9. The method of any of embodiments 1-8, wherein the glycosidase is a mannosidase.

Embodiment 10. The method of any of embodiments 1-9, wherein the reverse hydrolysis is aided by the substitution of the donor sugar with a leaving group.

Embodiment 11. The method of any of embodiments 1-10, wherein the leaving group is paranitrophenol.

Embodiment 12. The method of any of embodiments 1-11, wherein the mannosidase is a retaining enzyme and the donor sugar is substituted in the alpha-anomeric configuration.

Embodiment 13. The method of any of embodiments 1-12, wherein the retaining enzyme is Jack Bean Mannosidase.

Embodiment 14. The method of any of embodiments 1-13, wherein the mannosidase is an inverting enzyme, and the donor sugar is substituted in the beta-anomeric configuration.

Embodiment 15. The method of any of embodiments 1-14, wherein the inverting enzyme is a Class I ER exomannosidase.

The present invention, thus generally described, will be understood more readily by reference to the following example, which is provided by way of illustration and are not intended to be limiting of the present invention.

### EXAMPLE

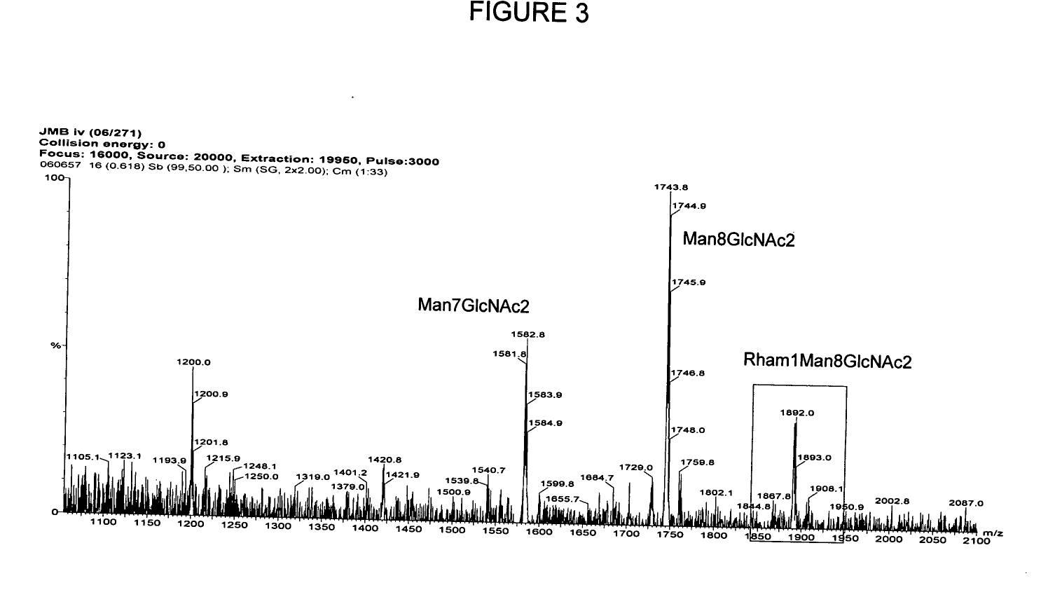

In one example, Man9GlcNAc2 is treated with an exomannosidase that cleaves the central D2 monosaccharide to yield Man8(B)GlcNAc2 (see FIG. 2). Subsequent reverse hydrolysis is performed using Jack Bean mannosidase (JBM) and paranitrophenyl-alpha-D-Rhamnose as a donor monosaccharide and to yield the novel compound Rham1Man8GlcNAc2. The progress of this reaction can be determined by MALDI-TOFF mass spectorometric analysis of the reaction products (FIG. 3).

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A pharmaceutical composition for inducing an immunogenic response against an oligo-D-mannose moiety of human immunodeficiency virus type 1 (HIV), the composition comprising:
(a) an effective amount of an antigen comprising an oligo-D-mannose moiety of HIV in which at least one D-mannose residue of the oligo-D-mannose moiety of HIV is substituted by at least one non-D-mannose monosaccharide residue; and
(b) a carrier.

2. The composition of claim 1, wherein the at least one non-D-mannose monosaccharide residue comprises a structural mimic of D-mannose.

3. The composition of claims 1, wherein the at least one non-D-mannose monosaccharide residue comprises a monosaccharide residue that is antigenic in the subject.

4. The composition of claim 1, wherein the at least one non-D-mannose monosaccharide residue comprises a monosaccharide residue that is non-natural to humans.

5. The composition of claim 1, wherein the at least one non-D-mannose monosaccharide residue comprises a monosaccharide residue selected from the group consisting of deoxy-monosaccharides, halo-substituted monosaccharides, nitro-substituted monosaccharides, amino-substituted monosaccharides, sulfo-substituted monosaccharides, and phosphor-substituted monosaccharides.

6. The composition of claim 5, wherein the deoxy-monosaccharides include rhamnose.

7. The composition of claim 1, wherein the antigen comprises a glycoprotein, a glycoconjugate scaffold, or a dendrimer.

8. The composition of claim 7, wherein the antigen is a glycoprotein comprising the substituted oligo-D-mannose moiety linked as an N-glycan.

9. The composition of claim 1, wherein the substituted oligo-D-mannose moiety has a formula selected from the group consisting of 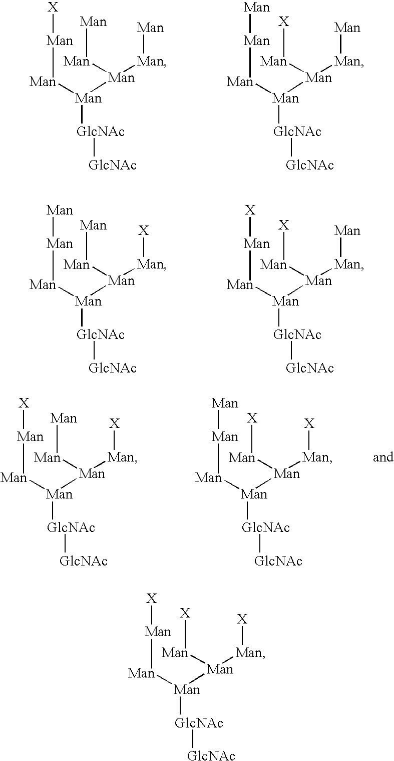
where “Man” is mannose, “GlcNAc” is N-acetylgalactosamine, and “X” is a non-D-mannose monosaccharide residue.

10. The composition of claim 9, wherein X is rhamnose.

11. The composition of claim 1, wherein the oligo-D-mannose moiety of HIV is present in HIV glycoprotein 120 (gp120) or HIV glycoprotein 41 (gp41).

12. The composition of claim 11, wherein the oligo-D-mannose moiety of HIV is the oligo-D-mannose moiety attached as an N-glycan at Asn332 or Asn392 of gp120.

13. The composition of claim 1, wherein the immunogenic response is a humoral response comprising production of antibodies that specifically bind the oligo-D-mannose moiety of HIV.

14. A method for inducing an immunogenic response against an antigen that comprises an oligo-D-mannose moiety, the method comprising administering the composition of claim 1 to a subject in need thereof.

15. A method for preparing an antigen for inducing an immunogenic response against HIV, the method comprising:
(a) treating an HIV oligo-D-mannose moiety with a first glycosidase to remove at least one D-mannose residue; and
(b) reacting the treated oligo-D-mannose moiety with at least one non-D-mannose monosaccharide residue in the presence of a second glycosidase to provide a substituted oligo-D-mannose moiety, thereby preparing the antigen for inducing the immunogenic response against HIV.

16. The method of claim 15, wherein the HIV oligo-D-mannose moiety is present in HIV gp120 or HIV gp41.

17. The method of claim 16, wherein the HIV oligo-D-mannose moiety is present in HIV gp120.

18. The method of claim 17, wherein the HIV oligo-D-mannose moiety is Man9GlcNAc2.

19. The method of claim 17, wherein the oligo-D-mannose moiety is the N-glycan attached to Asn332 or Asn392 of gp120.

20. The method of claim 15, wherein the first glycosidase is a mannosidase.

21. The method of claim 20, wherein the mannosidase is an exomannosidase.

22. The method of claim 15, wherein the second glycosidase is a mannosidase.

23. The method of claim 22, wherein the mannosidase is a retaining enzyme and the non-D-mannose monosaccharide residue has an alpha-anomeric configuration.

24. The method of claim 23, wherein the retaining enzyme is Jack Bean mannosidase.

25. The method of claim 22, wherein the mannosidase is an inverting enzyme and the non-D-mannose monosaccharide residue has a beta-anomeric configuration.

26. The method of claim 25, wherein the inverting enzyme is a class I ER exomannosidase.

27. The method of claim 15, wherein the at least one non-D-mannose monosaccharide residue comprises a structural mimic of D-mannose.

28. The method of claim 15, wherein the at least one non-D-mannose monosaccharide residue comprises a monosaccharide residue that is antigenic in humans.

29. The method of claim 15, wherein the at least one non-D-mannose monosaccharide residue comprises a monosaccharide residue that is non-natural in humans.

30. The method of claim 15, wherein the at least one non-D-mannose monosaccharide residue comprises a monosaccharide residue selected from the group consisting of deoxy-monosaccharides, halo-substituted monosaccharides, nitro-substituted monosaccharides, amino-substituted monosaccharides, sulfo-substituted monosaccharides, phosphor-substituted monosaccharides, and paranitrophenyl-substituted monosaccharides.

31. The method of claim 15, wherein the HIV oligo-D-mannose moiety is part of a glycoprotein, a glycoconjugate scaffold, or a dendrimer.

32. The method of claim 15, wherein the HIV oligo-D-mannose is part of a glycoprotein comprising the oligo-D-mannose moiety linked as an N-glycan.

33. The method of claim 15, wherein the substituted oligo-D-mannose moiety has a formula selected from the group consisting of Rham1Man8GlcNAc2, Rham1Man7GlcNAc2, and Rham1Man6GlcNAc2.

34. The method of claim 15, wherein the HIV oligo-D-mannose moiety is part of an HIV glycoprotein.

35. The method of claim 34, wherein the HIV glycoprotein is gp120 or gp41.

36. The method of claim 35, wherein the HIV glycoprotein is gp120 and the oligo-D-mannose moiety is attached as an N-glycan at Asn332 or Asn392.

37. The method of claim 15, wherein the substituted oligo-D-mannose moiety is Rham1Man8GlcNAc2.

38. The method of claim 15, wherein the non-D-mannose monosaccharide residue comprises a substitution at a hydroxyl position.

39. The method of claim 38, wherein the substitution comprises a leaving group.

40. The method of claim 39, wherein the leaving group is a paranitrophenyl group.

41. The method of claim 15, wherein the non-D-mannose monosaccharide residue comprises paranitrophenyl-alpha-D-rhamnose.

42. An antigen that comprises the substituted oligo-D-mannose moiety as prepared by the method of claim 15.

43. A pharmaceutical composition comprising the antigen of claim 42 and a carrier.

44. The composition of claim 43, wherein the antigen is present in the composition at a concentration effective for inducing an immunogenic response against HIV.
